(19) United States
(12) Patent Application Publication
(10) Pub. No.: US 2021/0370276 A1
(43) Pub. Date: Dec. 2, 2021

(54) Group 1 Metal Ion Content of Microporous Molecular Sieve Catalysts

(71) Applicant: ExxonMobil Chemical Patents Inc., Baytown, TX (US)

(72) Inventors: Larry L. Iaccino, Seabrook, TX (US); Xiaoying Bao, Houston, TX (US); Nitish Mittal, Houston, TX (US); Doron Levin, Highland Park, NJ (US); Wenyih F. Lai, Bridgewater, NJ (US); Jocelyn A. Gilcrest, Mullica Hill, NJ (US)

(21) Appl. No.: 17/285,350

(22) PCT Filed: Oct. 10, 2019
(86) PCT No.: PCT/US2019/055544

§ 371 Date: Apr. 14, 2021

Related U.S. Application Data
(60) Provisional application No. 62/752,553, filed on Oct. 30, 2018.

(51) Int. Cl. B01J 29/44; B01J 37/02; B01J 35/02; B01J 29/46; B01J 37/00

(52) U.S. Cl. CPC B01J 29/44; B01J 37/0201; B01J 35/026; C07C 5/333; B01J 37/0045; B01J 2229/30; B01J 2229/183; B01J 29/46

(57) ABSTRACT

A catalyst comprising a microporous crystalline aluminosilicate having a Constraint Index less than or equal to 12, a Group 1 alkali metal or a compound thereof and/or a Group 2 alkaline earth metal or a compound thereof, a Group 10 metal or a compound thereof, and optionally a Group 11 metal or a compound thereof; wherein the total amount of Group 1 and/or Group 2 metal is present at a ratio that is optimized for the desirable chemical conversion process.

### PRIORITY

This application claims priority to Provisional Application No. 62/752,553, filed Oct. 30, 2018, the disclosure of which is incorporated herein by reference.

### FIELD

The present invention relates to microporous molecular sieve preparation and in particular to the Group 1 metal content of such molecular sieves.

### BACKGROUND

Cyclopentadiene (CPD) is currently a minor byproduct of liquid fed steam cracking (i.e., naphtha and heavier feed). As steam cracking shifts more to lighter feeds (feed shift of existing facilities and new construction), less CPD is produced while demand is rising. High CPD price due to supply shortage limits the potential end product polymers. If additional CPD could be produced at unconstrained rates and potentially at a cost lower than recovery from steam cracking, then additional polymer product could be produced.

Metal containing microporous molecular sieves such as ZSM-5 crystal based catalysts have been discover to perform the cyclization of acyclic C5's. The desired catalyst for this process is one that maximizes the yield of cyclic C5's and minimizes loss of feed molecules to undesired byproducts. There is a need however to optimize such catalysts, and it has been found that the level of Group 1 metal ions, in particular, sodium and/or potassium, is important. The inventors have found certain optimum levels of desirable microporous molecular sieves.

The present application is related to U.S. Ser. No. 62/500,814 filed May 3, 2017, incorporated herein by reference.

### SUMMARY

Described herein is a catalyst comprising (or consisting of, or consisting essentially of) (i) a microporous crystalline aluminosilicate having a Constraint Index less than or equal to 12, (ii) a Group 1 alkali metal or a compound thereof and/or a Group 2 alkaline earth metal or a compound thereof, (iii) a Group 10 metal or a compound thereof, and optionally (iv) a Group 11 metal or a compound thereof; wherein the total amount of Group 1 and/or Group 2 metal is present at a ratio of at least 1.5 mols per mol of aluminum in the aluminosilicate. In any embodiment the Group 1/Group 2 ratio is at least 1.6, or 1.8, or 2.0, or 3.0; or within a range from 1.5, or 1.6, or 1.8, or 2.0, or 3.0 to 5.0, or 6.0, or 8.0, or 10.0.

Also described is a catalyst comprising (or consisting of, or consisting essentially of) (i) a microporous crystalline metallosilicate having a Constraint Index less than or equal to 12, (ii) a Group 1 alkali metal or a compound thereof and/or a Group 2 alkaline earth metal or a compound thereof, (iii) a Group 10 metal or a compound thereof, and optionally (iv) a Group 11 metal or a compound thereof; wherein the total amount of Group 1 and/or Group 2 metal present in the catalyst is at least 0.005 mols per mol of silica in the metallosilicate. In any embodiment the total amount of Group 1 and/or Group 2 metal present is at least 0.006, or 0.008, or 0.010, or 0.015, or 0.020 mols per mol silica; or within a range from 0.005, or 0.006, or 0.008, or 0.010, or 0.015, or 0.020 mols per mole silica to 0.05, or 0.06, or 0.07, or 0.08, or 0.09, or 0.10 mols per mole silica.

### DETAILED DESCRIPTION

The process to produce CPD often produces CPD as the primary product from plentiful C5 feedstock using a catalyst system to produce CPD while minimizing production of light (C1 to C4) byproducts. C5 feedstock may be virgin C5's (saturates, predominately normal pentane and isopentane, and/or methylbutane, with smaller fractions of cyclopentane and neopentane, and/or 2,2-dimethylpropane, from crude oil or natural gas condensate) or may be cracked C5'3s (above skeletal structures but in various degrees of unsaturation: alkanes, alkenes, dialkenes, alkynes) produced by refining and chemical processes: FCC, reforming, hydrocracking, hydrotreating, coking, and steam cracking. Lower hydrogen content (i.e., cyclic, alkenes, dialkenes) are preferred as the reaction endotherm is reduced and thermodynamic constraints on conversion are improved, but non-saturates are more expensive than saturate feedstock. Therefore a process to convert saturate C5's to CPD is most desired.

The process to convert saturated acyclic C5's to CPD needs a metallic functionality on the molecular sieve catalyst to affect the dehydrogenation and cyclization activity. This metallic functionality is preferably associated with an aluminosilicate crystal, preferably ZSM-5. The ZSM-5 crystals used in this catalyst are synthesized in the sodium form with final sodium levels on the crystal a function of the crystallization conditions and crystal recovery steps, e.g. washing.

It has now been discovered that the Group 1 metal, especially sodium, level on the ZSM-5 based catalyst impacts the performance of the finished catalyst used in a process for the cyclization of acyclic C5's. While not wishing to be bound by theory, we believe that sodium in excess of the amount of aluminum is required; this may be due to inefficiency in sodium titrating the aluminum sites, need for sodium to interact with silanol sites, and or the need for sodium to form zintl ions to help provide highly dispersed Pt. Other Group 1 and/or Group 2 metals could be included in conjunction with or instead of sodium.

Thus in any embodiment is a catalyst comprising (i) a microporous crystalline aluminosilicate having a Constraint Index less than or equal to 12, (ii) a Group 1 alkali metal or a compound thereof and/or a Group 2 alkaline earth metal or a compound thereof, (iii) a Group 10 metal or a compound thereof, and optionally (iv) a Group 11 metal or a compound thereof wherein the total amount of Group 1 and/or Group 2 metal is present at a ratio of at least 1.5 mols per mol of aluminum in the aluminosilicate. In any embodiment the Group 1/Group 2 ratio is at least 1.6, or 1.8, or 2.0, or 3.0; or within a range from 1.5, or 1.6, or 1.8, or 2.0, or 3.0 to 5.0, or 6.0, or 8.0, or 10.0.

Stated another way, in any embodiment is a catalyst comprising (i) a microporous crystalline metallosilicate having a Constraint Index less than or equal to 12, (ii) a Group 1 alkali metal or a compound thereof and/or a Group 2 alkaline earth metal or a compound thereof, (iii) a Group 10 metal or a compound thereof, and optionally (iv) a Group 11 metal or a compound thereof wherein the total amount of Group 1 and/or Group 2 metal present in the catalyst is at least 0.005 mols per mol of silica in the metallosilicate. In any embodiment the total amount of Group 1 and/or Group 2 metal present is at least 0.006, or 0.008, or 0.010, or 0.015, or 0.020 mols per mol silica; or within a range from 0.005, or 0.006, or 0.008, or 0.010, or 0.015, or 0.020 mols per mole silica to 0.05, or 0.06, or 0.07, or 0.08, or 0.09, or 0.10 mols per mole silica.

Stated another way, the catalyst has a Group 1 and/or Group 2 content of at least 0.1 wt %, or 0.2 wt %, or within a range from 0.1, or 0.2, to 0.5, or 0.8, or 1 wt %.

As used herein, a “catalyst” is a solid and/or liquid composition that is capable of catalyzing a chemical reaction preferably the conversion of acyclic hydrocarbons to cyclic hydrocarbons, and comprises at least a microporous molecular sieve, especially a microporous crystalline metallosilicate. The catalyst may also include one or more binders. To be used as a commercially viable catalyst, the microporous metallosilicate is combined with some binder, preferably a material that resists chemical reactions and physical changes due to heat, and further, can provide ridgid structure for the microporous metallosilicate. Thus, in any embodiment, the binder is selected from silica, titania, zirconia, alkali metal silicates, Group 13 metal silicates, carbides, nitrides, aluminum phosphate, aluminum molybdate, aluminate, surface passivated alumina, and mixtures thereof. In any embodiment, the catalyst is formed into one or more of the shapes of extrudates (cylindrical, lobed, asymmetric lobed, spiral lobed), spray dried particles, oil drop particles, mulled particles, spherical particles, and/or wash coated substrates; wherein the substrates may be extrudates, spherical particles, foams, microliths and/or monoliths.

As used herein “Group” refers to Groups of the Periodic Table of Elements as in HAWLEY'S CONDENSED CHEMICAL DICTIONARY, Thirteenth Edition (1997 John Wiley & Sons, Inc.).

As used herein, the “Constraint Index” is a measure of the extent to which a microporous molecular sieve (e.g., zeolites, aluminosilicates) provides controlled access of different sized molecules to its internal structure. For example, molecular sieves which provide a highly restricted access to and egress from its internal structure have a high value for the Constraint Index, and molecular sieves of this kind usually have pores of small size, e.g. less than 5 Angstroms. On the other hand, molecular sieves which provide relatively free access to the internal molecular sieves structure have a low value for the Constraint Index, and usually pores of large size.

A determination of the Constraint Index is made by continuously passing a mixture of an equal weight of n-hexane and 3-methylpentane over a small molecular sieves catalyst sample, approximately 1 gram or less, of catalyst at atmospheric pressure. A sample of the catalyst, in the form of pellets or extrudate, is crushed to a particle size about that of coarse sand and mounted in a glass tube. Prior to testing, the catalyst is treated with a stream of air at 1000° F. (538° C.) for at least 15 minutes. The catalyst is then flushed with helium and the temperature adjusted between 550° F. (288° C.) and 950° F. (510° C.) to give an overall conversion between 10% and 60%. The mixture of hydrocarbons is passed at 1 liquid hourly spaced velocity (i.e., one volume of liquid hydrocarbon per volume of catalyst per hour) over the catalyst with a helium dilution to give a helium to total hydrocarbon mole ratio of 4:1. After 20 minutes on stream, a sample of the effluent is taken and analyzed, most conveniently by gas chromatography, to determine the fraction remaining unchanged for each of the two hydrocarbons. The Constraint Index is then calculated using the following equation: Constraint Index=Log₁₀ (fraction of n-hexane remaining)/Log₁₀ (fraction of 3-methylpentane remaining).

The Constraint Index approximates the ratio of the cracking rate constants for the two hydrocarbons. Catalysts suitable for the present invention are those having a constraint index in the approximate range of 1 to 12. Constraint Index (CI) values for some typical catalysts are: Erinotite (38); ZSM-5 (8.3); ZSM-11 (8.7); ZSM-12 (2); ZSM-38 (2); ZSM-38 (4.5); synthetic Mordenite (0.5); REY (0.4); amorphous aluminosilicate (0.6).

As used herein, the “Alpha Value” of a molecular sieve catalyst is a measure of the cracking activity of that catalyst. Catalytic cracking activity is typically indicated by the weight percent conversion of hexane to lower boiling C1 to C5 hydrocarbons, while isomerization activity is indicated by weight percent conversion to hexane isomerization. The Alpha Value is an approximate indication of the catalytic cracking activity of the catalyst compared to a standard amorphous aluminosilicate catalyst obtained by co-gellation, 10% alumina, surface area of 420 m²/g, no cations in base exchanging solution. The cracking activity is obtained as a relative rate constant, the rate of n-hexane conversion per unit volume of oxides composition per unit time. This highly active aluminosilicate catalyst has an Alpha Value taken as 1. The experimental conditions of the test include heating the catalyst to a constant temperature of 538° C., and passing the hexane over the solid catalyst at that temperature at a variable flow rate to give contact times between 10 and 10⁻³ seconds. The tested particles should be smaller than 30 mesh in size, preferably 12 to 28 mesh. Alpha Values for some typical catalysts are: ZSM-5 with no cation exchange (38), and with H⁺ exchange (450); synthetic Faujasite exchanged in calcium ions (1.1), and exchanged in H(NH₄) (6,400).

So the inventive catalyst and methods of forming it can be further described by a number of features. For instance, in any embodiment the Group 1 and/or Group 2 metal is incorporated during aluminosilicate synthesis (crystallization). Also, in any embodiment the Group 1 and/or Group 2 metal level is controlled by the washing level after the synthesis of the aluminosilicate. This is effected as is known in the art such as flushing the crystalline solids with water through washing on a filter, and/or forming a slurry and decanting the dissolvate, and any other known means, by performing these steps for a longer or shorter time, and with greater or less volume of water or water solution. For instance, in any embodiment the Group 1 and/or Group 2 metal level is controlled by the Group 1 and/or Group 2 salt concentration in the wash liquid after the synthesis of the aluminosilicate.

In any embodiment, the Group 1 and/or Group 2 metal is incorporated by direct or sequential ion exchange after the synthesis of the aluminosilicate.

In any embodiment, the catalyst composition containing Group 1 and/or Group 2 has an Alpha Value (as measured prior to the addition of the Group 10 metal, and/or prior to the addition of the Group 11 metal) of less than 25, or 22, or 20, or 18, or 16, or 12, or 10.

Most any type of microporous crystalline metallosilicate that can catalyze the conversion of acyclic hydrocarbons, especially C4 to C10 hydrocarbons, into C4 to C10 cyclic hydrocarbons is desirable herein, In any embodiment the microporous crystalline metallosilicate described herein comprise a metallosilicate framework type selected from the group consisting of MWW, MFI, LTL, MOR, BEA, TON, MTW, MTT, FER, MRE, MFS, MEL, DDR, EUO, and FAU.

In any embodiment the microporous crystalline metallosilicate is an alumino silicate selected from the group consisting of Zeolite beta, mordenite, faujasite, Zeolite L, ZSM-5, ZSM-11, ZSM-30, ZSM-22, ZSM-23, ZSM-35, ZSM-48, ZSM-50, ZSM-57, ZSM-58, MCM-22 family material, and combinations thereof.

In one or more embodiments, the porous crystalline metallosilicate is crystalline aluminosilicate having a SiO₂/Al₂O₃ molar ratio greater than 25, or greater than 50, or greater than 100, or greater than 400, or greater than 1,000, or in the range from 25 to 2,000, or from 50 to 1,500, or from 100 to 1,200, or from 200 to 1000, or from 300 to 1000, or from 400 to 800.

Reference is made throughout this specification and claims to Group 1, Group 2, and Group 10 and 11 elements. In any embodiment the Group 1 alkali metal is selected from the group consisting of lithium, sodium, potassium, rubidium, cesium, and combinations thereof and/or the Group 2 alkaline earth metal is selected from the group consisting of beryllium, magnesium, calcium, strontium, barium, and combinations thereof.

In any embodiment the Group 10 metal is platinum and the source of platinum is selected from the group consisting of platinum nitrate, chloroplatinic acid, platinous chloride, platinum amine compounds, tetraamine platinum hydroxide, and combinations thereof.

In any embodiment the Group 11 metal is copper and the source of copper is selected from the group consisting of copper nitrate, copper nitrite, copper acetate, copper hydroxide, copper acetylacetonate, copper carbonate, copper lactate, copper sulfate, copper phosphate, copper chloride, and combinations thereof and/or the Group 11 metal is silver; and/or the source of silver is selected from the group consisting of silver nitrate, silver nitrite, silver acetate, silver hydroxide, silver acetylacetonate, silver carbonate, silver lactate, silver sulfate, silver phosphate, and combinations thereof.

As mentioned, the inventive catalyst may also comprise a binder. In any embodiment the binder is selected from silica, titania, zirconia, alkali metal silicates, Group 13 metal silicates, carbides, nitrides, aluminum phosphate, aluminum molybdate, aluminate, surface passivated alumina, and combinations thereof. In any embodiment the catalyst is formed into one or more of the shapes of extrudates (cylindrical, lobed, asymmetric lobed, spiral lobed), spray dried particles, oil drop particles, mulled particles, spherical particles, and/or wash coated substrates; wherein the substrates may be extrudates, spherical particles, foams, microliths and/or monoliths.

The inventive catalyst is useful for many types of catalysis, such as the conversion of acyclic hydrocarbons, especially C4 to C10 hydrocarbons, into C4 to C10 cyclic hydrocarbons. In any embodiment, the catalyst described herein is combined with acyclic C5's to form cyclic C5 compounds including cyclopentadiene. In any embodiment, the acyclic C5 conversion conditions include at least a temperature of 450° C. to 650° C., the molar ratio of an optional H₂ co-feed to the acyclic C5 feedstock is in the range of 0.01 to 3, the molar ratio of an optional light hydrocarbon co-feed to the acyclic C5 feedstock is in the range of 0.01 to 5, the acyclic C5 feedstock has a partial pressure in the range of 3 psia to 100 psia (21 to 689 kPa-a) at the reactor inlet, and the acyclic C5 feedstock has a weight hourly space velocity in the range from 1 hr⁻¹ to 50 hr⁻¹.

In any embodiment, the acyclic C5 conversion occurs in one of more reactors selected from radiantly heated tubular reactor, convectively heated tubular reactor, cyclically reheated fixed bed reactor, circulating fluid bed reactor, radiantly heated fluid bed reactor, convectively heated fluid bed reactor, adiabatic reactor and/or electrically heated reactor.

In any embodiment the catalyst is periodically rejuvenated and/or regenerated. This may be done in a vessel separate from the catalytic function of the catalyst, or in the same vessel as the primary catalytic function of the catalyst, such as the conversion of acyclic C5's to cyclic C5 compounds.

As such, a rejuvenation cycle is advantageously performed to produce a rejuvenated catalyst having restored or substantially restored catalyst activity, typically by removing at least a portion of the incrementally deposited coke material from the catalyst composition. Preferably, rejuvenated catalyst has activity restored to at least 50% of the activity of the catalyst prior to deactivation, more preferably at least 60%, more preferably at least 80%. Rejuvenated catalyst also preferably has restored or substantially restored catalyst selectivity, e.g., selectivity restored to at least 50% of the selectivity of the catalyst prior to deactivation, more preferably at least 60%, more preferably at least 80%. As used herein, “incrementally deposited coke” refers to the amount of coke that is deposited on the catalyst during a conversion cycle. Typically, a rejuvenation cycle is employed when the catalyst composition comprises >1 wt % incrementally deposited coke, such as >5 wt % incrementally deposited coke, or >10 wt % incrementally deposited coke. This is described in more detail in U.S. Ser. No. 62/500,795 filed May 2, 2017, incorporated herein by reference.

An article can be formed from cyclic C5 compounds described herein, alternatively in combination with pentene and/or pentadiene. In any embodiment the article is derived from a Diels-Alder reaction of the cyclic C5 compounds with a double bond containing compound. In any embodiment, the cyclic C5 compounds are selected from the group consisting of cyclopentadiene, dicyclopentadiene, cyclopentene, cyclopentane, norbornene, tetracyclodocene, substituted norbornenes, Diels Alder reaction derivatives of cyclopentadiene, cyclic olefin copolymers, cyclic olefin polymers, polycyclopentene, unsaturated polyester resins, hydrocarbon resin tackifiers, formulated epoxy resins, polydicyclopentadiene, metathesis polymers of norbornene or substituted norbornenes or dicyclopentadiene, and combinations thereof. In any embodiment, the article is selected from the group consisting of wind turbine blades, composites containing glass or carbon fibers, formulated adhesives, ethylidene norbornene, ethylene-propylene rubber, ethylene-propylene-diene rubber alcohols, plasticizers, blowing agents, solvents, octane enhancers, gasoline, and mixtures thereof.

### EXAMPLES

### Example 1: Synthesis of ZSM-5

A mixture with 22% solids was prepared from 52,800 g of DI water, 3,600 g of 50% NaOH solution, 156 g of 43% Sodium Aluminate solution, 4,380 g of n-propyl amine 100% solution, 120 g of ZSM-5 seed crystals, and 19,140 g of Ultrasil™ silica were mixed in a 30-gal pail container and then charged into a 30-gal autoclave after mixing. The mixture had the following molar composition (each component measured ±5% or less):

| | |
|---|---|
| SiO₂/Al₂O₃ | 470 |
| H₂O/SiO₂ | 10.73 |
| OH/SiO₂ | 0.16 |
| Na/SiO₂ | 0.16 |
| n-PA/Si | 0.25 |

The mixture was mixed and reacted at 210° F. (99° C.) at 150 rpm for 48 hours. The resulting reaction slurry was discharged and stored in a 30-gal pail container. No flashing to remove excess n-propylamine was performed. The reaction slurry was then flocced, washed/filtered, and dried for use. The XRD pattern of the as-synthesized material showed the typical pure phase of ZSM-5 topology. SEM of the as-synthesized material showed that the material was composed of mixture of distinct crystals with mixed size of 0.5 to 1.5 micron. The resulting ZSM-5 crystals had a Na content of 0.57 wt % (0.66 wt % after correcting for % solids) and a Na/Al ratio of 3.67. The zeolite has an Alpha Value from 5 to 10, and a Constraint Index from 3 to 5.

### Example 2: Synthesis of ZSM-5

A mixture with 22% solids was prepared from 8,800 g of DI water, 600 g of 50% NaOH solution, 26 g of 43% Sodium Aluminate solution, 730 g of n-propyl amine 100% solution, 40 g of ZSM-5 seed crystals, and 3,190 g of Ultrasil silica were mixed in a 5-gal pail container and then charged into a 5-gal autoclave after mixing. The mixture had the following molar composition (each component measured ±5% or less):

| | |
|---|---|
| SiO₂/Al₂O₃ | 470 |
| H₂O/SiO₂ | 10.73 |
| OH/SiO₂ | 0.16 |
| Na/SiO₂ | 0.16 |
| n-PA/Si | 0.25 |

The mixture was mixed and reacted at 230° F. (110° C.) at 350 rpm for 48 hours. The resulting reaction slurry was discharged and stored in a 5-gal pail container. No flashing to remove excess n-propyl amine was performed. The reaction slurry was then flocced, washed/filtered, and dried for use. The XRD pattern of the as-synthesized material showed the typical pure phase of ZSM-5 topology. SEM of the as-synthesized material showed that the material was composed of mixture of distinct crystals with size of 0.3 micron. The resulting ZSM-5 crystals had a Na content of 0.18 wt % (0.2 with correction of solids) and a Na/Al ratio of 1.03. The zeolite has an Alpha Value from 5 to 10, and a Constraint Index from 3 to 5.

### Example 3: Synthesis of ZSM-5

A 20% solids mixture containing DI water, 50% NaOH solution, 43% Sodium Aluminate solution, n-propyl amine 100% solution, ZSM-5 seed crystals, and Ultrasil silica was charged to an autoclave. The reaction mixture had the following molar composition (each component measured ±5% or less):

| | |
|---|---|
| SiO₂/Al₂O₃ | 470 |
| H₂O/SiO₂ | 12.3 |
| OH/SiO₂ | 0.16 |
| Na/SiO₂ | 0.16 |
| n-PA/Si | 0.25 |

The mixture was mixed and reacted at 220° F. (110° C.) at 75 rpm for about 40 hours. Residual n-propyl amine in the mother liquor was removed by flashing at 240° F. after completion of crystallization. The resulting slurry was then transfer to a decanter for floccing and decantation. The flocced slurry was then filtered, washed and dried. The XRD pattern of the as-synthesized material showed the typical pure phase of ZSM-5 topology. SEM of the as-synthesized material showed that the material was composed of mixture of distinct crystals with size of 0.5 micron. The resulting ZSM-5 crystals had a Na content of about 0.49 wt % with correction of % solids, Na/Al (molar ratio) of about 2.54, and a carbon content of 1.84 wt %. The zeolite has an Alpha Value from 5 to 10, and a Constraint Index from 3 to 5.

### Example 4: Impregnation of Pt on ZSM-5

A sample of the ZSM-5 crystal prepared in Example 1 was calcined for 9 hours in nitrogen at 900° F. The atmosphere was then gradually changed to 1.1, 2.1, 4.2, and 8.4% oxygen in four stepwise increments. Each step was followed by a thirty minute hold. The temperature was increased to 1000° F., the oxygen content was increased to 16.8%, and the material was held at 1000° F. for 6 hours. The impregnated extrudate results in the catalyst. After cooling, 0.50 wt % Pt as measured by XRF was added via incipient wetness impregnation using an aqueous solution of tetraamine platinum nitrate. The catalyst was dried in air at 121° C. (250° F.) and then calcined in air for three hours at 350° C. (660° F.).

### Example 5: Impregnation of Pt on ZSM-5

A sample of the ZSM-5 crystal prepared in Example 2 was calcined for 9 hours in nitrogen at 900° F. The atmosphere was then gradually changed to 1.1, 2.1, 4.2, and 8.4% oxygen in four stepwise increments. Each step was followed by a thirty minute hold. The temperature was increased to 1000° F., the oxygen content was increased to 16.8%, and the material was held at 1000° F. for 6 hours. The impregnated extrudate results in the catalyst. After cooling, 0.49 wt % Pt as measured by XRF was added via incipient wetness impregnation using an aqueous solution of tetraamine platinum nitrate. The catalyst was dried in air at 121° C. (250° F.) and then calcined in air for three hours at 350° C. (660° F.).

### Example 6: Preparation of 40:60 ZSM-5:SiO₂ Extrudate

A sample of the ZSM-5 crystal prepared in Example 3 was used to prepare a 40 wt % ZSM-5:60 wt % silica extrudate. 40 parts by weight of zeolite were mulled with 60 parts by weight of silica. The silica was equally supplied by Ultrasil silica and by Ludox HS-40. Sufficient water was added to produce a mull mix of 58 wt % solids. The material was extruded into 1/20″ cylinders and then dried overnight at 121° C. (250° F.). After drying, the extrudate was calcined for in air at 650° C. for 45 minutes.

### Example 7: Preparation of Reduced Sodium ZSM-5: SiO₂ Extrudate

A sample of the extrudate prepared in Example 6 was exchanged with ammonium nitrate at various concentrations at room temperature for 1 hour followed by washing with DI water, drying at 121° C. (250° F.) and then calcined in air at 538° C. (1000° F.) for 1 hour. Calcined samples were analyzed for sodium content using ICP. Table 1 summarizes the examples herein.

TABLE 1

| | Ammonium Nitrate | |
|---|---|---|
| Sample | Concentration (N) | Na, wt % |
| 6 | Not Exchanged | 0.48 |
| 7A | 0.025 | 0.29 |
| 7B | 0.075 | 0.15 |
| 7C | 0.15 | 0.13 |
| 7D | 0.5 | 0.08 |

### Example 8: Impregnation of Pt on 40:60 ZSM-5:SiO₂ Extrudates

Samples of extrudates prepared in Examples 6 and 7 were impregnated with Pt using Platinum Tetraamine hydroxide to a target of 0.5% Pt based on the weight of ZSM-5 crystal (0.2% Pt based on weight of extrudate). The impregnated extrudate results in the catalyst. After impregnation, samples were dried at 121° C. (250° F.) and then calcined at 475° C. for 4 hours.

### Example 9: Conversion of n-Pentane

A sample of the catalyst prepared in Example 4 and 5 were evaluated for performance in the conversion of n-pentane to CPD. The catalyst (0.25 g crushed and sieved to 20-40 mesh) was physically mixed with SiC (8 g, 40-60 mesh) and loaded into a 0.28″ ID, 18″ long stainless steel reactor. The catalyst bed was held in place with quartz wool and the reactor void space was loaded with metal inserts. The reactor was loaded onto the unit and pressure tested to ensure no leaks. The catalyst was dried for 1 hour under helium (200 mL/min, 3045 psig, 250° C.) then reduced for 4 hours under H₂ (200 mL/min, 45 psig, 500° C.). The catalyst was then tested for performance with feed of n-pentane, H₂, and balance helium, 5.0 psia C₅H₁₂, 1.0 molar H₂:C₅H₁₂, and 45 psig total. The catalyst was initially de-edged at 550° C. for 8 hours at WHSV=15 h⁻¹ then tested at 575° C. at WHSV=30 h⁻¹.

The average yield of cyclic C5 products (CPD, cyclopentene and cyclopentane) (C %) measured at 15 hours on stream (7 hours after end of de-edging) were 32% and 19% for the catalysts of Example 4 (Na/Al=3.65) and Example 5 (Na/Al=1.03), respectively.

### Example 10: Conversion of n-Pentane

A sample of the catalysts prepared in Example 6 and 7 were evaluated for performance in the conversion of n-pentane to CPD. The catalyst (0.625 g crushed and sieved to 20 to 40 mesh) was physically mixed with high-purity SiC (40-60 mesh) and loaded into a 9 mm ID, 13 mm OD, 19″ long quartz reactor. The amount of SiC was adjusted so that the overall length of the catalyst bed was 6 in. The catalyst bed was held in place with quartz wool and the reactor void space was loaded with coarse SiC particles. The reactor was loaded onto the unit and pressure tested to ensure no leaks. The catalyst was dried for 1 hour under helium (145 mL/min, 30 psig, 250° C.) then reduced for 4 hours under H2 (270 mL/min, 30 psig, 500° C.). The catalyst was then tested for performance with feed of n-pentane, H2, and balance helium, 3.3 psia C5H12, 1.0 molar H2:C5H12, and 30 psig total. The catalyst was tested at 575° C. at a n-pentane WHSV=15 h-1. Average yield of cyclic C5 products (CPD, cyclopentene and cyclopentane) measured at 16 hours on stream is shown in Table 2.

TABLE 2

| Sample | C5 Yield (wt. %) |
|---|---|
| 6 | 19.7 |
| 7A | 17.6 |
| 7B | 7.3 |
| 7C | 2.1 |
| 7D | 1.0 |

### Fluidizable Particles

It is contemplated that the zeolites crystals such as but not limited to those discussed above could be formed into fluidizable particles. Such materials could be formed by deagglomerating the sodium zeolite crystals The sodium zeolite crystals could then be mixed with matrix material which could be a mixture of inorganic materials such as silica, alumina, titania or zirconia and clays such as kaolin and bentonite to form an aqueous slurry. The matrix could be peptized. Any surface acidity of alumina in the binder is either minimized or controlled through the addition of alkaline metals, alkaline earth metals or a source of phosphorus to the spray dry slurry. The slurry could be dried such as by spray drying and then calcined to form a fluid powder of, for example, less than 200 microns in diameter. Optionally, the acidity of the alumina could be controlled by post-treatment with sources of alkaline metals or alkaline earth metals such as by impregnation. The fluidizable particles can then be treated with sources of desired metals such as platinum, platinum and silver or platinum and copper to form metal containing, formulated sodium ZSM-5.

As used herein, “consisting essentially of” for the catalyst means that the catalyst may include minor amounts of ingredients not named, but does not include any other ingredients, or made from any other essential calcining steps, not named that would influence its catalytic activity towards conversion of acyclic alkanes to cyclic alkanes to an analytically significant extent such as ±1, 2, or 5 wt % of a final product or overall rate.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the invention have been illustrated and described, various modifications can be made without departing from the spirit and scope of the invention.

1. A catalyst comprising (i) a microporous crystalline aluminosilicate having a Constraint Index less than or equal to 12, (ii) a Group 1 alkali metal or a compound thereof and/or a Group 2 alkaline earth metal or a compound thereof, (iii) a Group 10 metal or a compound thereof, and optionally (iv) a Group 11 metal or a compound thereof; wherein the total amount of Group 1 and/or Group 2 metal is present at a ratio of at least 1.5 mols per mol of aluminum in the aluminosilicate.

2. The catalyst of claim 1, wherein the Group 1/Group 2 ratio is at least 1.6.

3. A catalyst comprising (i) a microporous crystalline metallosilicate having a Constraint Index less than or equal to 12, (ii) a Group 1 alkali metal or a compound thereof and/or a Group 2 alkaline earth metal or a compound thereof, (iii) a Group 10 metal or a compound thereof, and optionally (iv) a Group 11 metal or a compound thereof; wherein the total amount of Group 1 and/or Group 2 metal present in the catalyst is at least 0.005 mols per mol of silica in the metallosilicate.

4. The catalyst of claim 3, wherein the total amount of Group 1 and/or Group 2 metal present is at least 0.006 mols per mole silica.

5. The catalyst of claim 1, wherein Group 1 and/or Group 2 metal is incorporated during aluminosilicate synthesis (crystallization).

6. The catalyst of claim 1, wherein Group 1 and/or Group 2 metal level is controlled by the washing level after the synthesis of the aluminosilicate.

7. The catalyst of claim 1, wherein Group 1 and/or Group 2 metal level is controlled by the Group 1 and/or Group 2 salt concentration in the wash liquid after the synthesis of the aluminosilicate.

8. The catalyst of claim 1, wherein Group 1 and/or Group 2 metal is incorporated by direct or sequential ion exchange after the synthesis of the aluminosilicate.

9. The catalyst of claim 3, wherein the catalyst composition containing Group 1 and/or Group 2 has an Alpha Value (as measured prior to the addition of the Group 10 metal, and/or prior to the addition of the Group 11 metal) of less than 25.

10. The catalyst of claim 3, wherein the microporous crystalline metallosilicate comprises a metallosilicate framework type selected from the group consisting of MWW, MFI, LTL, MOR, BEA, TON, MTW, MTT, FER, MRE, MFS, MEL, DDR, EUO, and FAU.

11. The catalyst of claim 3, wherein the microporous crystalline metallosilicate is an alumino silicate selected from the group consisting of Zeolite beta, mordenite, faujasite, Zeolite L, ZSM-5, ZSM-11, ZSM-30, ZSM-22, ZSM-23, ZSM-35, ZSM-48, ZSM-50, ZSM-57, ZSM-58, MCM-22 family material, and combinations thereof.

12. The catalyst of claim 3, wherein the Group 1 alkali metal is selected from the group consisting of lithium, sodium, potassium, rubidium, cesium, and combinations thereof; and/or the Group 2 alkaline earth metal is selected from the group consisting of beryllium, magnesium, calcium, strontium, barium, and combinations thereof.

13. The catalyst of claim 3, wherein the Group 10 metal is platinum and the source of platinum is selected from the group consisting of platinum nitrate, chloroplatinic acid, platinous chloride, platinum amine compounds, tetraamine platinum hydroxide, and combinations thereof.

14. The catalyst of claim 3, wherein the Group 11 metal is copper and the source of copper is copper nitrate, copper nitrite, copper acetate, copper hydroxide, copper acetylacetonate, copper carbonate, copper lactate, copper sulfate, copper phosphate, copper chloride, or combinations thereof; and/or the Group 11 metal is silver, wherein the source of silver silver nitrate, silver nitrite, silver acetate, silver hydroxide, silver acetylacetonate, silver carbonate, silver lactate, silver sulfate, silver phosphate, or combinations thereof.

15. The catalyst of claim 3, also comprising a binder.

16. The catalyst of claim 15, wherein the binder includes silica, titania, zirconia, alkali metal silicates, Group 13 metal silicates, carbides, nitrides, aluminum phosphate, aluminum molybdate, aluminate, surface passivated alumina, or combinations thereof.

17. The catalyst of claim 3, wherein the catalyst is formed into one or more of the shapes of extrudates (cylindrical, lobed, asymmetric lobed, spiral lobed), spray dried particles, oil drop particles, mulled particles, spherical particles, and/or wash coated substrates; wherein the substrates may be extrudates, spherical particles, foams, microliths and/or monoliths.

18. The catalyst of claim 17, wherein the catalyst has a Group 1 and/or Group 2 content of at least 0.1 wt %.

19. (canceled)

20. (canceled)

21. (canceled)

22. (canceled)

23. (canceled)

24. (canceled)

25. (canceled)
